(19) United States
(12) Patent Application Publication
(10) Pub. No.: US 2002/0151521 A1
(43) Pub. Date: Oct. 17, 2002

(54) Universal antiviral composition

(76) Inventors: Peter A. Burke, Skillman, NJ (US); Stephen L. Coulter, Yardley, PA (US)

Correspondence Address: JOHN LEZDEY, SUITE A, 1409 NORTH FT HARRISON, CLEARWATER, FL, 33755, US

(21) Appl. No.: 09/903,289

(22) Filed: Jul. 11, 2001

Related U.S. Application Data
(63) Continuation-in-part of application No. 09/281,391, filed on Mar. 30, 1999.

(51) Int. Cl. A61K 31/728; A61K 31/726; A61K 31/715; A61K 31/14; A61K 9/14

(52) U.S. Cl. 514/54; 424/487; 514/643

(57) ABSTRACT

There is provided an universal antiviral composition in the form of a lotion, foam or gel that is non-irritating. The composition contains an effective amount antimicrobicidal agent, an acidic buffer and wound healing agent so that the pH is an 7. The composition of the invention can be used in connection with packaged

This application is a continuation-in-part of application Serial No. 09/281,391 filed Mar. 23, 1999.

### FIELD OF THE INVENTION

The present invention relates to an universal antiviral composition which can be used intranasally, topically on the epidermis, mucosal membranes or any devices intended to contact body parts as well as in the packaging of condoms. More particularly, there is provided a non-irritating composition, which is viricidal, spermicidal, and microbicidal, which can be used prophylactically.

### BACKGROUND OF THE INVENTION

The population around the world is constantly coming in contact with bacteria and viruses through social and/or accidental means. Those infected by the virus or bacteria can in many cases be treated with conventional antibiotics. However, some of the bacteria or viruses such as hepatitis, HIV or herpes result in serious illnesses. Furthermore, many of the bacteria or viruses mutate and become resistant to the antibiotic. Therefore, it has become necessary to kill the virus or bacteria before the party is infected. It is therefore desirable to prophylactically protect a host from contamination by the bacteria or virus.

The bacteria or virus easily invades a host through mucous membranes or openings in the skin. The skin is generally a good barrier against invasion by microorganisms and washing can eliminate microorganisms. However, there are times when a party who has come into contact with a microorganism cannot wash or treat an area of the body so that it is necessary to provide anti-microbial protection before and/or during contact in an area where infection can occur.

Due to the variety and complexity of the potential outcomes of sexual activity, including pregnancy, disease and discomfort, agents which can be used alone or in conjunction with condoms will require functionality, that cannot reasonably be derived from a singular entity. For example, standard lubricants with detergents, specifically Nonoxynol-9 (N-9), provide protection against pregnancy and a variety of sexually transmitted diseases (STDs). The detergents also have a drawback in that in high concentrations and/or high frequency of use, they have potential to cause irritation of the mucosal tissue.

It is desirable to improve the efficacy of the agents while reducing the deleterious side effects. The primary focus is on strategies that can be used with or without condoms, but must address both pregnancy and disease prevention. There are other agents effective as spermicides and mircobicides including other detergents, buffering agents, and even selective inorganic agents. Combinations of these can increase the breadth of activity against STDs. For example, the use of buffering agents is highly effective against pregnancy, but of limited value in protection against STDs.

A third factor in the design of effective therapies includes the incorporation of agents selected to not only reduce possible irritation caused by one or more of the other agents, but to even ameliorate existing irritation.

One additional factor is to specifically include strategies directly targeting HIV. Due to the significance of HIV infection, specific agents should also be added to bolster the efficacy. Some of the agents already being considered for the amelioration of irritation are also effective binding agents for the same cell receptors that HIV targets. In addition, HIV-specific agents, while still in the early stages of testing, can be incorporated in future formulations.

In order for a composition to be capable of universal use it is required that it meet at least the following characteristics.

1. The composition is anti-microbial

2. The composition can be used in sensitive areas, for example mucosal membranes, therefore it must be non-imitating.

3. The composition must form a barrier.

When used in connection with packaged condoms it is essential that none of the ingredients can cause degradation of the condom and that the condom maintains a long shelf life. Latex condoms are especially sensitive to many chemicals especially oils and some detergents/soaps, as well as is well known in the field. Incompatible chemicals can weaken latex films (decreased tensile properties) and compromise the condoms.

The art of packaging condoms is well known and involves the use of metal foils and polymeric films as the packaging material.

Advantageously, the pH is adjusted for each site of use. There exists a specific need for improving methods and compositions for preventing sexually transmitted diseases (STDs) and unwanted pregnancies both with and without condoms. This objective should be realized by maintaining normal and protective vaginal flora that play a role in the prevention of vaginitis, vaginosis, and urinary tract infections. The present invention is also based on the realization that a method providing rapid and reliable control of vaginal pH could provide a highly effective, yet highly physiologic means to achieve these goals.

The most effective contraceptive methods (sterilization, intrauterine devices, and contraceptive hormones) provide no protection against STDs. Barrier contraceptive methods such as condoms, diaphragms, and vaginal spermicides help prevent STD transmission by interposing a mechanical or chemical barrier between the female and the male, most importantly between the uterine cervix and the glans and urethral orifice of the penis. This barrier action is effective because secretions from the sites (cervical mucus and semen) are most important sources of STD pathogens and also because these sites are the most susceptible targets for many STD pathogens. However, current barrier methods suffer from poor acceptance and therefore poor efficacy, and/or have irritation potential. Some male and female condoms are cumbersome to use and may reduce sexual pleasure and intimacy. Vaginal spermicides can erode the mucosa if used too frequently, and even with infrequent use, can disrupt the protective normal vaginal flora.

The pH of a healthy vagina is mildly acidic (pH 3.5-4.5) and this acidity is thought to be generated by the production of lactic acid by lactobacilli, which form a major component of the healthy vaginal flora. Together with other factors, this acid pH is widely recognized to prevent overgrowth of undesirable microbes (Candida, harmful anaerobes, and bacteria that may cause urinary tract infections) and encourages the continued dominance of lactobacilli which, in addition to mild acidity, provide other protective mechanisms such as production of hydrogen peroxide.

It is also known that sperm is inactivated by the mild acidity of the healthy vagina, the acid substances have been used as home made vaginal contraceptives for centuries. More recently it has been recognized that many sexually transmitted diseases. Pathogens, Niesseria gonorrhea (McCutchan 1977), Treponema palladium, Haemophilus ducreyi, and most or all enveloped SmD viruses (Kempf 1991, Martin 1985) including herpes simplex virus, cytomegalovirus, and human immunodeficiency virus, are also inhibited or inactivated by a mid pH. However, semen contains a potent alkaline buffering capacity that neutralizes the vaginal acidity for a period of many hours after intercourse. This alkaline buffering capacity enables sperm to swim from the vagina into the cervix and upper genital tract.

Unfortunately, STD pathogens in genital secretions can also exploit this period of neutral vaginal pH, since it allows time for them to reach and infect their target cells. If this semen-induced neutralization of vaginal acidity could be promptly and reliably overcome, both contraception and STD prevention could be achieved by a method that closely mimics the normal physiological state of the vagina.

In addition, the elevated pH also allows certain strains of Staphylococcus aureus to produce shock toxin I, whereas production of this toxin is completely inhibited at acidic pH<5.0 (Schlievert 1983). Thus, loss of protective acidity may result in staphylococcal toxic shock syndrome, candida vaginitis, bacterial vaginosis, or unriniy tract infection.

U.S. Pat. No. 4,912,093 to Michaeli, which is herein incorporated by reference, discloses sulfated polysaccharides, which can be used in the present invention.

U.S. Pat. Nos. 5,824,658 and 5,604,200 disclose wound healing compositions which contain hyaluronic acid.

U.S. Pat. No. 5,617,877 to Moench et al. discloses a contraceptive composition comprising a spennicide and cellulosic delivery system over which the present invention provides an advantage.

### SUMMARY OF THE INVENTION

The present invention provides an universal antiviral composition for use topically and in mucosal areas of the body. The composition comprises:

a) an effective amount of an antimicrobial agent;

b) a buffer in an effective amount for maintaining the pH of the composition at a proper pH for the area of the application, and 4iz c) an effective wound healing and diffusion impedence amount of compound such as a polysaccharide selected from the group consisting of dextran sulfate, chondroitin sulfate, pentosan polysulfate, a hyaluronate, glucosamino glycan, sulfated and sulfonated polymers, cellulosics, and synthetic polymers with similar functionalities.

Advantageously, when the composition is used in mucosal areas the pH is about 3.5-5.0.

The composition is especially useful in packaging latex condoms.

The composition can be formulated as a créme, lotion, gel, foam, and the like.

It is therefore an object of the invention to provide an universal antiviral composition for use on different parts of the body.

It is a further object of the invention to provide both an anti-viral and spermicidal composition, which can prevent STD.

It is yet another object of the invention to provide a composition to prevent S'ID for use with packaged condoms and the like.

These objects and other advantages of the invention will be better understood from a reading of the description of the preferred embodiments and the examples. Description of the Preferred Embodiments The antimicrobial or antiviral compounds, which may be used in the invention include nonoxynol.9, N-docosanol (a nonionic surfactant), benzalkonium chloride (a cationic surfactant), sulfated polysaccharides such as dextrin sulfate, pentosan polysulfate, carrageenan, and the like, binding antagonists such as PROCEPT@, non-nucleoside reverse transcriptase inhibitors such as nevirapine, VC7$1, acyclovir, idoxyumidine, ribavirin, vidarabin, rimantadine, phenol mercuric acetates, ricinolec acid, and the like, antiviral serine protease inhibitors such as aprotinin, N-alpha-tosyl-LI-ysyl-chloromethyl ketone (TACK) and 4 acetamidophenyl 4-guanidinobenzoate.

Binding agents such as sulfated polysaccharides bind to mucosal proteins and block the CD-4 cell receptor, which the AIDS virus attached itself for infectivity.

Hyaluronates, such as hyaluronic acid, are polysaccharides which have been found to have multiple utilities, especially when used in combination with many spermicidal and/or microbicidal compounds. The hyaluronates provide antI-MV absorption protection, they are anti-irritants of mucous tissue, they promote tissue healing and ameliorate ulceration. The hyaluronates include hyaluronic acid, it's salts, esters, amides, and other derivatives.

The hyaluronates as a result of their penetration into tissue also provide a barrier against viral infiltration. In combination with a surfactant the penetration is quicker.

The aqueous gels of the invention, especially those containing highly carboxylated polymers have a further utility as a sexual lubricant capable of preventing the exchange or transmission of SID pathogens and sperm during sexual activity.

When formulated as a lubricant, the compositions can be applied to external genitalia as well as internal mucosal surfaces to reduce microtrauma resulting from inadequate lubrication but will prevent transmission of viable STD pathogens through traumatized, diseased or healthy skin or mucosa. The presence of hyaluronic acid as well as a sene protease inhibitor such as alpha 1-antitpsin provides the additional feature of wound healing and reduction of inflammation. Hyaluronic acid penetrates or is absorbed into the epidermis so as to act as a barrier to prevent penetration of SID pathogens if there is any injury. The hyaluronic acid component is further usefull to promote wound healing if there is no sexual activity.

The compositions according to the invention may be presented in all forms normally used for topical application, in particular in the form of aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase (O/W) or vise versa (W/O), or of suspensions or emulsions of soft, semi-solid consistency of the creme or gel type, or alternatively of microemulsions, of tnicrocapsules, of microparticles or of vesicular dispersions to the ionic or non-ionic type. These compositions are prepared according to the standard methods.

They may also be used for the scalp in the form of aqueous, aqueous-alcoholic solutions, or in the form of cremes, gels, emulsions, or foams or alternatively in the form of aerosol compositions also containing a propellant agent under pressure.

The amounts of the different constituents of the compositions according to the invention are those traditionally used in the cosmetics and pharmaceutical fields.

The compositions may also be packaged in the form of an aerosol composition containing a propellant agent under pressure.

When the composition of the invention is an emulsion, the proportion of the fatty phase can range from 5% to 80% by weight, and preferably from 5% to 50by weight relative to the total weight of the composition. The emulsifiers and coemulsifiers used in the composition in emulsion form are chosen from those traditionally used in cosmetics.

The emulsifier and the coemulsifier are present in the composition in a proportion ranging from 0.3% to 30% by weight, and preferably 0.5% to 30% or, better still, from 0.5% to 20%, by weight relative to the total weight of the composition. The emulsion can, in addition, contain lipid vesicles. 4 When the composition of the invention is a gel or solution, the fatty phase can represent more than 90% of the total weight of the composition.

In a known manner, the composition of the invention may also contain adjuvants which are customary in the pharmaceutical or cosmetic field, such as hydrophilic or lipophilic gelling agents, hydrophilic or lipophilic active agents, preservatives, antioxidants, solvents, perfimes, and fillers. The amounts of these different adjuvants are those traditionally used in the pharmaceutical or dermatological field, and are, for example, from 0.01% to 10% of the total weight of the composition. Those adjuvants, depending on their nature, may be introduced into the fatty phase, into the aqueous phase and/or into lipid sperules, for use in shampoos, body lotions, hand lotions and the like.

Fatty alcohols, fatty acids (stearic acid) and waxes (paraffin, carnuba, beeswax) may also be used in the invention, glycerol, stearate, polysorbate 60 and the PEG-6 PEG-32 glycol stearate mixture sold under the name Tefosei® 63 by the company Gattefosse may be mentioned as examples.

As hydrophilic gelling agents, carboxyvinyl polymers (Carbomer®), acrylic copolymers such as acrylate/alkylacrylate copolymers, polyacryla mides, polysaccharides such as hydroxypropylcellulose, clays and natural gumns may be mentioned, and as lipophilic gelling agents, modified clays such as bentones, metal salts, of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, essential fatty acids, ceramides and essential oils may be used. These agents add extra moisturizing or skdn softening features when utilized.

The compositions of the invention may include plant or herbal extracts. For example, there may be utilized extracts of Paraguay tea, Kola and Guarana, which provide a source of methylxanthines, saponius, tannins, and glycosides that have been shown to be anti-infiammatory and can be used to treat or prevent irritations, The extract of Paraguay tea is known as “ate extract” and is described in the “International Cosmetic Ingredient Dictionary”, 5 Edition. Mate extract is commercially available in combination with extracts of Kola and Guarana, which is sold by Cosmetic Ingredient Resources of Stamford, CT. under the trademark “QUENCHI.”Each of mate extract, serine protease inhibitor and aloe vera extract are known to provide anti-inflammatory activity. The anti-elastase and anti-tryptase activity of the protease inhibitor has been shown to provide a synergistic effect in treating skin inflammations.

Up to 10% by weight of zinc salts can be used to prevent irritations when required. Suitable zinc salts include zinc gluconate, zinc acetate, zinc chloride, etc.

A surfactant can be included in the composition so as to provide deeper penetration of the ingredients. Many surfactants also possess anti-microbial activity.

The surfactants which can be used in the present compositions when nonoxynol-9 or the other named anti-viral agents are the water soluble anionic, nonionic, amphoteric, zwitterionic or cationic surfactants.

Suitable anionic surface active agents include, for example, alkali meW salts of long chain fatty sulphates, alkali metal ether sulphates derived from alcohols and alkali phenols, alkali metal sulpho-succinates, alkali metal sarcosinates and alkali metal taurides. Suitable cationic surface active agents include quaternary ammonium bromides and chlorides containing a long chain alkyl such as, for example, cetrimide. Suitable amphoteric surface active agents include so called “betaine ” type and imidazoline type surface active agents.

Preferred anionic surfactants include alkyl dimethylamine oxides having 12 to 25 carbon atoms such as N, N-dimethyl-l-tetradecanamine oxide and N,N-dimethyl-1-octadecananime oxide, sodium lauroyl sarcosinate, diphenyl ether sulfonates such as the alkali metals salts of hexadecyl diphenyl ether disulfonic acid, dodecyl diphenyl ether disulfonic and decyl diphenyl ether disulfonic acid, preferably Cio-Cig alkylbenzene sulfonates. Commercially available anionic surfactants, which may be used include Ufaryl DL80, DL85, and DL90 of Unger Fablikker which are mixtures of CIO-C₁₃ inear sodium alkylbenzene sulfonate. Udet 950 of De Soto, Calsoft F90 of Pilot Corporation (a CIO-CI₃ sodium linear alkylayl sulfonate), Witconate 9OP of Witco Corporation (a C,₂ sodium alkylaryl sulfonate containing 1.7% free oil and 3.0% S0₄), Nansa HS 80PF of Albright & Wilson Ltd. and Stepan Agent S-150965 of Stepan Corporation (a C13 calcium dodecylbenzene sulfonate) are also suitable.

Nonionic surfactants, which can be used in practicing the present invention can be of three basic types - the alkylene oxide condensates, the amides and the semi-polar nonionics.

The alkylene oxide condensates are broadly defined as compounds produced by the condensation of alkylene oxide groups (hydrophilic in nature) with an organic hydrophobic compound, which can be aliphatic or alkyl aromatic in nature. The length of the hydrophilic polyoxyalkylene radical, which is condensed with any particular hydrophobic group can be readily adjusted to yield a water soluble compound having the desired degree of balance between hydrophilic and hydrophobic elements.

Examples of such alkylene oxide condensates include:

1. The condensation products of aliphatic alcohols with ethylene oxide. The alkyl chain of the aliphatic alcohol can either be straight or branched and generally contains from about 8 to about 22 carbon atoms. Examples of such ethoxylated oxide include the condensation product of about 6 moles of ethylene oxide with 1 mole of tridecanol, myristyl alcohol the condensation product of ethylene oxide with coconut fatty alcohol wherein the coconut alcohol is a mixture of fatty alcohols with alkyl chains varving fron about 10 to 14 carbon atoms and wherein the condensate contains about 6 moles of ethylene oxide per mole of alcohol and the condensation product of about 9 moles of ethylene oxide with the above-described coconut alcohol. Examples of colmmercially a vailable nonionic suffactants of this type include Tergitol 15-S-9 marketed by the Shell Chemical Company and Kyro EOB marketed by The Proctor & Gamble Company.

2. The condensation products of ethylene oxide with a hydrophobic base formed by the condensation of propylene oxide with propylene glycol. The hydrophobic portion of these compounds, has a molecular weight of from about 1500 to 1800 and of course include certain of the commercially available Pluronic surfactants marketed by the Wyandotte Corporation.

3 The condensation products of ethylene oxide with the product resulting from the reaction of propylene oxide and ethylene diamine. The hydrophobic base of these products consists of the reaction of propylene oxide and ethylene diamine and excess propylene oxide said base having a molecular weight of from about 2500 to about 3000. This base is condensed with ethylene oxide to the extent that the condensation product contains from about 40% to about 80% weight of the polyoxvethylene and has a molecular weight of from about 5,000 to about 11,000. Examples of this type of nonionic surfactant include certain of the commercially available Tetronic compounds marketed by the Wyandotte Chemicals Corporation.

Examples of the Ode type of nonionic surfactants include the ammonia, monoethanol and diethanol amid(:s of fatty acids having acyl moieties of from about 8 to about 18 carbon atoms. These acyl moieties are normally derived from naturally occurring glycerides, e.g. coconut oil, palm oil, soybean oil and tallow, but can be derived synthetically, e.g. by the oxidation of petroleum, or by hydrogenation of carbon monoxide by the Fischer-Tropsch process.

Examples of the semi-polar type of nonionic surfactants are the amine oxides, phosphine oxides and sulphoxides. These materials are described more fully in U.S. Pat. No., 3,819,528, Berry, issued Jun. 25, 1974, and incorporated herein by reference.

Amphoteric surfactants, which can be used in practicing the present invention can be broadly described as derivatives of aliphatic amines which contain a long chain of about 8 to about 18 carbon atoms and an anionic water-solubilizing group, e.g. carboxyl, sulfo and sulfato. Examples oil compounds falling within this definition are sodium 3-dodecylamino-propionate, sodium-3-dodecylamino propane sulfonate, and dodecyl dimethylammonium hexanoate.

Zwitterionic surfactants, which can be used in practicing the present invention can be broadly described as internally-neutralized derivatives of aliphatic quaternary ammonium and phosphonium and tertiary sulfonium compounds, in which the aliphatic radical can be straight chain oil branched, and wherein one of the aliphatic substituents contains an anionic water solubilitizing group, e.g. carboxyl, sulfo, sulfato, phosphate, or phosphono.

Cationic surfactants, which can be used in practicing the present invention include stearyl dimethyl benzyl ammonium chloride, coconut dimethyl benzyl ammonium chloride, cetyl pyridinium chloride, and ce V trimethyl ammonium chloride.

Particularly, preferred surfactants for use herein are sodium and potassium alkyl naphthalene sulfonates having one or two alkyl group containing about 1 to about 6 carbonates. RSO3M, wherein R is a primary or secondaiy alkyl group containing from about 8 to about 22 carbon atoms (preferably about 12 carbon atoms), and M is an alkali metal.

The preferred spermicidal/microbicidal composition of the invention especially for use in packaged condoms comprises:

a) about 0.1 to 10% by weight of detergent,

b) about 0.5 to 5% by weight of buffering agent;

c) about 0 to 10% by weight of an anti-irritant;

d) about 0.1 to 5% by weight of cellular binding agents;

e) an HIV specific inhibitor; and

f) the remainder being fillers.

In the composition, the hyaluronates, particularly hyaluronic acid has multiple roles. It is a buffering agent, spermicide, a cellular binding agent and provides an effective barrier upon absorption to prevent HIV penetration.

The detergents also have multiple roles such as being a surfactant, a spermicide and a HIV inhibitor.

The spermicidal/microbicidal compositions of the invention are especially useful in combination with a lubricant base that is used alone or in combination with a condom.

The preferred lubricants include water, propylene glycol, glycerin, polyethylene glycol and a silicone.

Thickeners of up to 50% by weight of the lubricant base can be utilized. Suitable thickeners include cellulosic polymers, natural gums, synthetic polymers and silicon dioxide.

The preferred cellulosic thickeners, which are used in an amount up to about 3% by weight include methylcellulose, carboxymethylcellulose, hydroxypropylceliulose, hyaluronic acid, sulfonated and sulfonated polysaccharides.

The preferred natural gums, which can be used in amounts up to about 5% by weight include xantham gum, aliginates and gelatin.

The preferred synthetic polymers, which can be used include Carbopol®, polyacrylic acid, polymethacrylic acid, hydroxyethyl methacrylate, polyacrylamide, polyvinyl pyrrolidone and polyvinyl alcohol.

The following examples illustrating the compositions of the invention are not intended to limit the scope of the invention. The amounts indicated are by weight percent unless otherwise noted.

### EXAMPLE 1

A gel is prepared by admixing the following ingredients:

| | Ingredient | Wt. % |
|---|---|---|
| 1. | Propylene Glycol | 43.44 |
| 2. | Carbopol | 2.10 |
| 3. | Dipropylene glycol | 10.00 |
| 4. | Xantham Gum | 0.15 |
| 5. | Ethoxydiglycol | 15.00 |
| 6. | Dimethylisosorbide | 10.00 |
| 7. | Ascorbic Acid | 2.00 |
| 8. | Chloroxylenol | 0.20 |
| 9. | Linoleamidopropyl PG-diammonium chloride phosphate | 1.50 |
| 10. | Glycereth 4,5 Lactate | 2.00 |
| 11. | Aloe Vera Gel | 2.00 |
| 12. | Aprotinin | 2.00 |
| 13. | Benzalkonium chloride | 0.50 |
| 14. | Hyaluronic acid | 8.00 |
| 15. | Cocamidopropyl PG-diammonium chloride phosphate | 1.00 |

Ingredients 1 and 2 are mixed to disperse and form a gel. About 80% of ingredient 3 is mixed with ingredient 4, added to the gel and slightly heated with admixture. The balance of 3 is mixed with ingredients 5-10 and added to the gel.

Ingredients 11-15 are then admixed then added to the gel at 38 degrees C. After mixing, the pH is adjusted to about 4 and then the gel is brought to room temperature. The composition can be used for packaging condoms.

Example 2

A topical lotion is prepared by admixing the following ingredients:

| Ingredient | Wt % |
|---|---|
| Propylene Glycol Stearate | 9.50 |
| Isocetyl alcohol | 5.00 |
| PEG-100 Stearate | 1.20 |
| Hyaluronic acid | 2.00 |
| Methyl paraben | 0.20 |
| Propylene glycol | 13.10 |
| Sorbitan palmitate | 0.60 |
| Octoxynol-9 | 6.00 |
| Mate extract | 0.50 |
| Water | g.s. |
| | 100% |

Example 3

A gel is prepared by admixing the following ingredients.

| Ingredient | Wt % |
|---|---|
| Carbopol 940 | 4.10 |
| Xantham Gum | 0.15 |
| Propylene glycol | 47.94 |
| Dipropylene glycol | 10.00 |
| Ethoxydiglycol | 15.00 |
| Dimethylisosorbide | 10.00 |
| Hyaluronic acid | 8.00 |
| Nonoxynol-9 | 0.05 |
| Aprotinin | 3.76 |
| Buffer | 2.00 |
| | 100% |

The pH of the gel is about 4. The gel can be used in packaging condoms.

Example 4

An antimicrobial lubricant is prepared as follows:

A. Lubricant Base

| Ingredient | % w/w |
|---|---|
| Propylene glycol | 80 |
| Carragenen gum | 3 |
| Lubrajel ® | 17 |
| | 100% |

B. Preparation of Composition

To the admixture of the lubricant base from Part A is added 5% by weight hyaluronic acid, 3% by weight of nonoxynol-9 and 3% by weight of polyacrylic acid. The mixture is then brought to a pH of 4. If desired, 0.5% of zinc acetate or mate extract may added.

The composition can be used in combination with condoms which are to be packaged.

Example 5

A lubricating composition having anti-microbial and spermicidal activity is prepared by mixing the following ingredients:

| Ingredient | % w/w |
|---|---|
| Hyaluronic acid | 5.0 |
| Octoxynol-9 | 0.5 |
| Zinc gluconate | 0.5 |
| Acyclovir | 0.5 |
| Carragenen gum | 3.0 |
| Carbopol | 40.0 |
| Water | g.s. |
| | 100% |

The composition can be used as in connection with packaged condoms or in combination with other ingredients to form an antimicrobial lotion.

What is claimed is:

1. An universal antiviral composition, which comprises:
a) an effective amount of a microbicidal agent to prevent sexually transmitted diseases;
b) an acidic buffer in an effective amount for maintaining the composition at a pH not greater than 5 for the site of administration; and
c) an effective amount of wound healing and diffusion impedance compound which is a member selected from the group consisting of dextran sulfatechondroitin sulfate, pentosan polysulfate, glucosamine glycan, sulfated and sulfonated polymers and a hyaluronate.

2. The composition of claim 1 wherein said hyaluronate is hyaluronic acid.

3. The composition of claim 1 wherein said microbicidal agent is selected from the group consisting of nonoxynol-9, octoxynol-9, acyclovir, benzalkonium chloride and aprotinin.

4. The composition of claim 1 wherein said microbicidal agent is spermicidal.

5. The composition of claim 1 including an acrylic acid polymer.

6. The composition of claim 1 including lubricants.

7. The composition of claim 1 including about 0.1 to 10% by weight of detergent.

8. The composition of claim I wherein said composition is for use in a non- mucosal area and includes a cellulosic binder.

9. The composition of claim 1 in a gel.

10. The composition of claim 9 wherein said lubricant base is a member selected from the group consisting of water, polyethylene glycol, propylene glycol,glycerine and a silicone.

11. A spermicidal and microbicidal composition comprising:
a) about 0.1 to 10% by weight of a spermicidal and microbicidal detergent;
b) about 0.5 to 5% by weight of a buffering agent; and
c) about 0.1 to 5% by weight of a member selected from the groupconsisting of dextran sulfate, chondroitin sulfate, pentosan polysulfate and a hyaluronate, said composition having a pH less than 5 and in the form of a lotion, creme, or a gel.

12. The composition of claim 11 comprising:
a) about 0.1 to 10% by weight of nonoxynol-9;
b) about 0.5 to 5% of polyacrylic acid; and
c) about 0.1 to 5% of hyaluronic acid, said composition including a lubricating base and having a pH of about 5.

13. A package comprising a condom and the composition of claim 1 within said package.

14. A package comprising a condom and the composition of claim 3 within said package.

15. A package comprising a condom and the composition of claim 5 within said package.

16. A package comprising a condom and the composition of claim 11 within said package.

17. In a packaged condom the improvement, which consists of said condom ing a spermicidal and antimicrobial composition comprising:
a) about 0.1 to 10% by weight of a sperinicidal and microbicial t;
b) about 0,05 to 5% by weight of a buffering agent;
c) about 0.1 to 5% by weight of a member selected from the group ing of dextran sulfate, chondroitin sulfate, pentosan polysulfate and a hyaluronate;
d) the remainder being fillers.

18. The packaged condom of claim 17, wherein said detergent is nonoxynol-9, buffering agent comprises polyacrylic acid, said hyaluronate is hyaluronic acid.
